(19) United States
(12) Patent Application Publication
(10) Pub. No.: US 2011/0124659 A1
(43) Pub. Date: May 26, 2011

(54) CYCLOALKANE DERIVATIVE

(75) Inventor: Hironori KINOSHITA, Osaka (JP)

(73) Assignee: Dainippon Sumitomo Pharma Co., Ltd., Osaka (JP)

(21) Appl. No.: 12/945,402

(22) Filed: Nov. 12, 2010

Related U.S. Application Data
(60) Provisional application No. 61/261,864, filed on Nov. 17, 2009.

(51) Int. Cl. A61K 31/496; C07D 417/14; A61P 25/00; A61P 25/18

(52) U.S. Cl. 514/254.04; 544/368

(57) ABSTRACT

The present invention relates to a novel cycloalkane derivative which has a useful psychotropic action with less side effect.

### CROSS REFERENCE TO RELATED APPLICATION

This application is a utility application and claims the benefit of U.S. Provisional Application No. 61/261,864, filed Nov. 17, 2009, the complete disclosure of which is incorporated herein by reference.

### TECHNICAL FIELD

The present invention relates to a novel cycloalkane derivative and an acid addition salt thereof which are useful as a psychotropic compound. In more detail, the compound of the present invention is useful as a medicament for treating, for example, schizophrenia, geriatric psychosis, bipolar disorder, depression, neurosis, senile dementia and associated symptoms thereof.

### BACKGROUND ART

Patent References 1-3 disclose some cycloalkane derivatives which have psychotropic action.

The compounds disclosed in Patent References 1-2 are different from the derivatives of the present invention, in the structures of R¹ and R² attached at C5 or C6 of the bicycloheptane dicarboximide ring in formula [1].

In addition, the compounds disclosed in Patent Reference 3 are different from the derivatives of the present invention, since the compounds of Patent Reference 3 have no bicycloheptane dicarboximide ring in the present formula [1].

Furthermore, psychotropic drugs which have been currently used can be accompanied with some disorders such as side effects in CNS, extrapyramidal disorder (e.g. catalepsy), oversedation, as well as cognitive decline. Consequently, the disorders of such drugs have been a serious problem in clinical field (Non-patent Reference 1).

### Prior Art

### Patent Reference

Patent Reference 1: JP-5 (1993)-17440 A (U.S. Pat. No. 5,532,372 a)
Patent Reference 2: JP-8 (1996)-333368 A
Patent Reference 3: WO 96/14297

### Non-Patent Reference

Non-patent Reference 1: The Pharmacological basis of therapeutics, A. Goodman Gilman, L. S. Goodman et al, New York (1985) P 387, GENDAI IRYO, 22, P22, (1990)

### DISCLOSURE OF INVENTION

### Problem to be Solved by the Invention

The purpose of the present invention is to provide a good psychotropic drug which has less side effects. Especially, the purpose is to provide a psychotropic drug which exhibits an excellent effect for improving a broad spectrum of schizophrenia such as positive symptom, negative symptom, and cognitive symptom, while almost preventing abnormal electrocardiogram, weight gain, increased blood glucose, etc., i.e. the desired drug is very safe and could be administered for a long term.

### Means to Solve the Problem

The present inventor has extensively studied to reach the above object and then has found that the novel cycloalkane derivatives of the present invention exhibit the desired pharmacological actions and further reduce the side effects. Based upon the new findings, the present invention has been completed.

The present invention relates to the following:

A first aspect concerns a cycloalkane derivative of formula [1]:

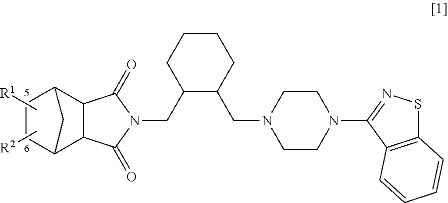

wherein

R¹ and R² are attached at C5 or C6 of the bicycloheptane dicarboximide ring (at the same carbon atom or different carbon atoms), and are independently hydrogen, hydroxyl, hydroxymethyl or —CH(C₁₋₃ alkyl)OH,

provided that when one of R¹ and R² is hydrogen, the other is neither hydrogen nor hydroxyl, or

when R¹ and R² are attached at the same carbon atom, they may be combined to form ═O, ═CH₂ or ═CH—C₁₋₃ alkyl, or a stereoisomer or an acid addition salt thereof.

A second aspect concerns a cycloalkane derivative of the first aspect wherein the compound of formula [1] is the following stereo structure:

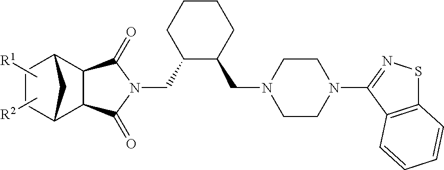

or a stereoisomer or an acid addition salt thereof.

A third aspect concerns a cycloalkane derivative of either the first or second aspect wherein R¹ and R² is independently hydroxyl, hydroxymethyl or —CH(C₁₋₃ alkyl)OH, or a stereoisomer or an acid addition salt thereof.

A fourth aspect concerns a cycloalkane derivative of the first or second aspect wherein one of R¹ and R² is hydrogen, and the other is hydroxymethyl or —CH(C₁₋₃ thereof.

A fifth aspect concerns a cycloalkane derivative of the first or second aspect wherein R¹ and R² are attached at the same carbon atom and combined to form ═O, ═CH₂ or ═CH—C₁₋₃ alkyl, or a stereoisomer or an acid addition salt thereof.

A sixth aspect concerns a cycloalkane derivative of the first aspect wherein the compound of formula [1] is the following structure:

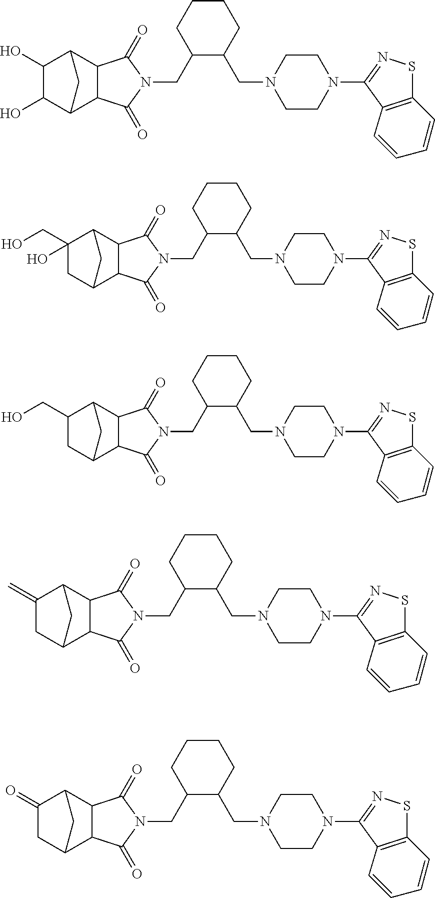

or a stereoisomer or an acid addition salt thereof.

A seventh aspect concerns a cycloalkane derivative of the first aspect wherein the compound of formula [1] is the following structure:

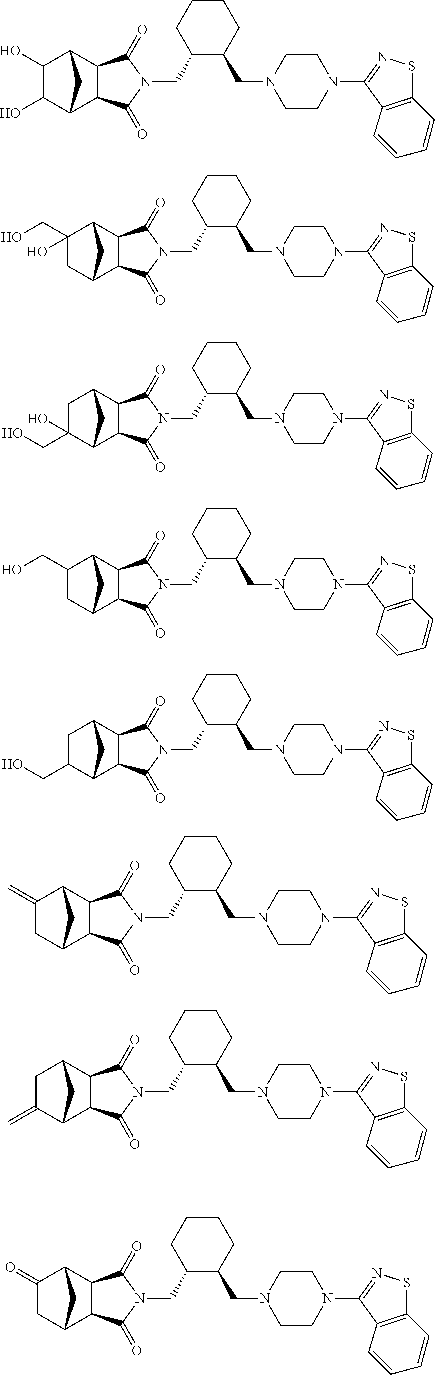

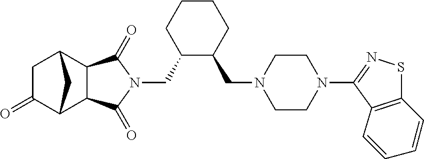

or a stereoisomer or an acid addition salt thereof.

A eighth aspect concerns an antipsychotic agent comprising the cycloalkane derivative of any one of the first through seventh aspects or a stereoisomer or an acid addition salt thereof.

A ninth aspect concerns a method for treating psychosis comprising administering an effective amount of the cycloalkane derivative of any one of the first through seventh aspects or a stereoisomer or an acid addition salt thereof to a mammal in need thereof.

A tenth aspect concerns the use of the cycloalkane derivative of any one of the first through seventh aspects or a stereoisomer or an acid addition salt thereof in preparation of an antipsychotic agent.

### BRIEF DESCRIPTION OF THE FIGURE

FIG. 1 lists compounds with their associated calculated 50% inhibition concentrations (IC₅₀ [μM]) based on data obtained from Test 1, Test 2 and Test 3 that were fitted with the Hill equation.

### BEST MODE FOR CARRYING OUT THE INVENTION

The present compounds can exist as a hydrate and/or solvate and hence also include such hydrate and/or solvate thereof.

The present compounds have plural asymmetric carbon atoms. Accordingly, the present compounds may include any possible stereoisomer(s) and/or optical isomer(s) as well as any combination(s) thereof, unless otherwise indicated.

The term “C₁₋₃ alkyl” used herein means methyl, ethyl, n-propyl or isopropyl (2-propyl).

The site number in the bicycloheptane dicarboximide used herein is shown below, i.e. C5 and C6 mean positions 5 and 6, respectively.

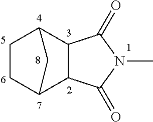

The acid addition salt used herein includes an addition salt with a pharmaceutically acceptable inorganic acid or organic acid. The salt with an inorganic acid includes, for example, hydrochloride, hydrobromide, hydroiodide, sulfate, nitrate, phosphate, etc. and preferably hydrochloride. The salt with an organic acid includes, for example, acetate, oxalate, citrate, malate, tartrate, maleate, fumarate, etc.

### Preparation

The present compounds [1] can be prepared, for example, by the method shown in the following schemes.

Every compound having stereoisomers of the invention can be separated to a single isomer by column chromatography (with a chiral column or a non-chiral column), optical resolution, or other general resolution method.

### Side Chain (1)

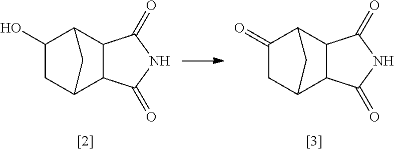

The carbonyl compound [3] can be prepared by oxidizing the hydroxyl compound [2]. The oxidation used herein can be carried out under a general oxidation condition, for example, using a metal oxidizing agent such as chromium or an organic oxidizing agent such as dimethylsulfoxide. The preferable oxidation is chromium oxidation, especially using the Jones reagent. The solvent used in the oxidation reaction is preferably selected to meet the oxidation condition, for example, acetone is preferable for the Jones reagent, and a halogen solvent such as dichloromethane and chloroform is preferable for organic oxidizing agent. With regard to the temperature condition, room temperature is preferable for the oxidation using a metal oxidizing agent, and a range of −78° C. to room temperature is preferable for the oxidation using an organic oxidizing agent.

### Side Chain (2)

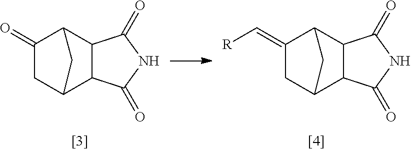

wherein R is hydrogen or C₁₋₃ alkyl.

The alkenyl compound [4] can be prepared by reacting the carbonyl compound [3] with a phosphorus ylide such as Wittig reagent. The solvent used herein includes ethers such as tetrahydrofuran and 1,2-dimethoxyethane, alcohols, and DMSO. The base used herein includes an alkyllithium such as butyllithium, a silazane such as sodium hexamethyldisilazide (SHMDS), sodium amide, sodium ethoxide, lithium diisopropylamide (LDA), DBU, etc. The reaction temperature is −78° C. to reflux temperature of the reaction solvent, and preferably around 50° C.

### Side Chain (3)

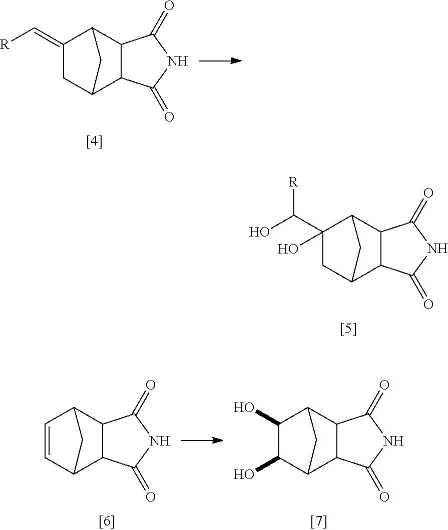

wherein R is as defined above, and bold solid lines denote a relative configuration.

The diol compound [5] can be prepared by oxidizing the alkenyl compound [4]. In a similar manner, the compound [7] can be prepared from the compound [6]. The oxidizing agent used herein includes osmium (VIII) oxide, potassium permanganate, etc., and preferably osmium (VIII) oxide. The agent for facilitating oxidation used herein includes H₂O₂, t-BuOOH, NaClO₃, N-methylmorpholine-N-oxide, etc. and preferably N-methylmorpholine-N-oxide. The solvent used herein includes a mixture of acetone/water or t-BuOH/water, etc. The reaction temperature is 0° C. to reflux temperature of the reaction solvent, and preferably room temperature.

### Side Chain (4)

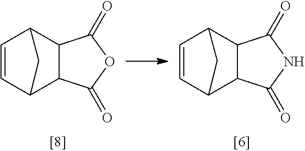

The above compound [6] can be prepared by heating under reflux the compound [8] in conc. aqueous ammonia.

### Side Chain (5)

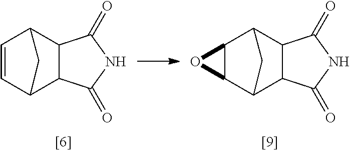

wherein bold solid lines denote a relative configuration.

The epoxy compound [9] can be prepared by oxidizing the alkenyl compound [6]. The oxidizing agent used herein includes a peracid such as hydrogen peroxide, performic acid, peracetic acid, trifluoroperacetic acid, perbenzoic acid, and m-chloroperbenzoic acid, and oxone, and preferably m-chloroperbenzoic acid. The solvent used herein includes a chlorinated solvent such as dichloromethane and chloroform. The reaction temperature is 0° C. to reflux temperature of the reaction solvent, and preferably room temperature.

### Side Chain (6)

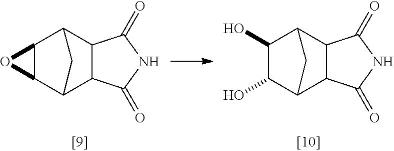

wherein bold solid lines and hash line denote a relative configuration.

The diol compound [10] can be prepared by acid-hydrolyzing the epoxy compound [9]. The acid used herein includes hydrochloric acid, sulfuric acid, etc. The reaction temperature is 0° C. to reflux temperature of the reaction solvent, and preferably around 60° C.

### Side Chain (7)

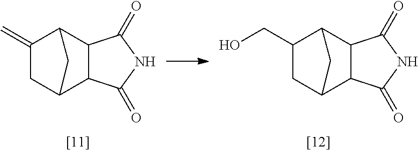

The alcohol compound [12] can be prepared by the hydroboration-oxidation of the alkenyl compound [11]. The solvent used herein includes ethers such as ethyl ether, tetrahydrofuran and dioxane. The hydroboronating agent used herein includes borane, disiamylborane, thexylborane, and 9-borabicyclo[3.3.1]nonane. The reaction temperature is 0° C. to reflux temperature of the reaction solvent, and preferably room temperature.

### Side Chain (8)

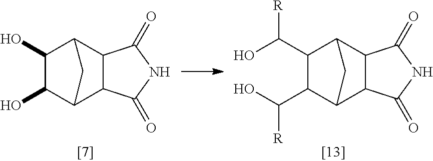

wherein R is as defined above, and bold solid lines denote a relative configuration.

The diol compound [7] is oxidized under the above condition (Side chain (1)) to form a carbonyl compound. The carbonyl compound is subjected to the above condition (Side chain (2)) to form an alkenyl compound. The alkenyl compound can be oxidized under the above condition (Side chain (7)) to prepare the carbon-chain-elongated compound [13].

### Compound [1]

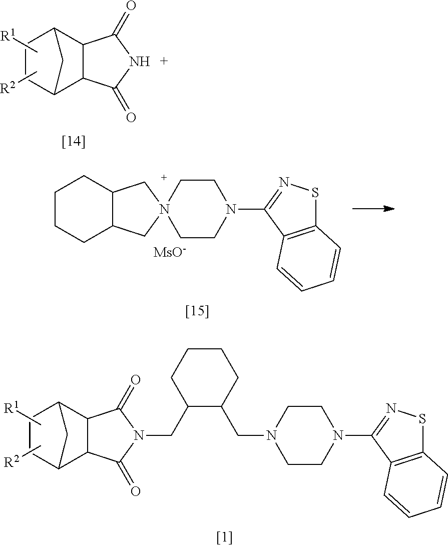

wherein R¹ and R² are as defined above.

The present compound [1] can be prepared by reacting the bicyclo compound [14] with the ammonium salt [15] in the presence of a base. The solvent used herein includes an alcohol, dimethylformamide, acetonitrile, toluene, etc. and preferably a mixture of dimethylformamide and toluene. The reaction temperature is room temperature to reflux temperature of the reaction solvent. The base used herein includes an inorganic base such as potassium carbonate, sodium carbonate and cesium carbonate.

When both of compounds [14] and [15] are single stereoisomers, a single stereoisomer of compound [1] can be prepared.

Diastereomer mixture of compound [1] can be separated to a single diastereomer by column chromatography, recrystallization or other general resolution method.

The present compound can be administered orally or parenterally in the medical use. Namely, the compound can be orally administered as a generally-used dosage form such as powder, granule, tablet, capsules, syrup, and suspension, or parenterally administered as an injection form such as solution, emulsion, and suspension thereof. And it can be rectally administered as a suppository. Furthermore, it can be intravesically administered as a solution. The above-mentioned drug form can be prepared by formulating the present compound with conventional additives such as carrier, excipient, binder, stabilizer, and diluent. In the case of injections, for example, acceptable buffer, solubilizer, and isotonic agent can be also used. In the case of the above-mentioned oral formulation or suppository, the present compound may be contained preferably in 0.1-70% (w/w) per the composition. The dosage and the frequency of administration depend on various conditions such as target disease, symptom, age and body weight of a subject, type of formulation, and manner of administration. In general, the present compound can be administered in a dosage of 0.1-2000 mg, preferably 1-200 mg per a day for an adult, and once to several times (e.g. twice to 4 times) a day.

The compounds of the invention are useful for treating psychosis, in more detail as follows.

The compounds of the invention exhibit high affinity for subtypes of various/plural receptors, for example, dopamine D₂ receptor, serotonergic receptor such as serotonin 5-HT_{1A}, serotonin 5-HT₂ and serotonin 5-HT₇, and α2 noradrenergic receptor.

It has been well known that D2 receptor antagonistic action in a subtype of dopaminergic receptor is strongly correlated with psychotic effect (see: e.g. Seeman, Pharmacol. Rev., 32, 229 (1981)). And also, it has been reported that 5-HT₂ receptor antagonistic action in a subtype of serotonergic receptor is useful for antipsychotic effect (see: e.g. Janssen et al., J. Pharm. Exper. Ther., 244, 685 (1988)). In addition, it has been known that antagonistic action of serotonin 5-HT₇ which is one of serotonergic receptors can improve cognitive function and lead to antidepressive action (J. Med. Chem. 2007, 50, 4214-4221, TRENDS in Pharmacological Sciences Vol. 25 No. 9, 481-486). Especially, D2 receptor antagonistic action can control positive symptoms of schizophrenia (e.g. hallucination, delusion), while 5-HT₂ receptor antagonistic action can contribute to improve negative symptoms of schizophrenia (e.g. indifference, social withdrawal). In addition, it has been suggested that 5-HT₂ receptor antagonistic action can decrease some side effects in the extrapyramidal tract which often arises in a maintenance therapy of schizophrenia using D2 receptor antagonist.

Further, it has been reported that antagonistic action of 5-HT_{1A} receptor which is a subtype of other serotonergic receptors is correlated with antianxiety (see: e.g. Titeler, Biochem. Pharmacol., 36, 3265 (1987)).

Accordingly, the compounds of the invention have psychotropic actions such as antipsychotic action, antianxiety, and antidepressive action, which are useful, for example, as a medicament for treating schizophrenia, geriatric psychosis, bipolar disorder, depression, neurosis, senile dementia and associated symptoms thereof, etc.

### EXAMPLES

Hereinafter, the present invention is further illustrated by Reference examples, Examples, and Tests, but should not be construed to be limited thereto. In the following schemes, the substituent connected to the middle of the bond in the cycloalkane ring is meant to be attached to either end of the bond or attached to the both as a mixture thereof. The triangular solid lines and hash lines thereof denote an absolute configuration and the bold solid lines and hash lines thereof denote a relative configuration.

### Reference Example 1

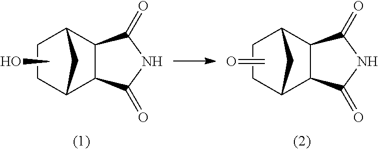

To a solution of Compound (1) as a starting material (4.21 g, 23 mmol, prepared by the method described in Patent Reference 2) in acetone (20 ml) was added the Jones reagent [prepared by adding conc. H₂SO₄ (2.3 ml) to a solution of chromium (VI) oxide (2.7 g, 27 mmol) in water (10 ml)] dropwise at ice temperature under nitrogen atmosphere. The mixture was stirred at ice temperature for hours, then isopropanol was added thereto, and the mixture was further stirred at room temperature for 30 minutes. The reaction mixture was filtrated through Celite and washed out with acetone. The filtrate was concentrated in vacuo to a small amount, but not dried up. The given residue was stood at room temperature, then the precipitated crystal was collected through a filter. The given crystal was washed with a mixture of acetone/hexane (1:1) to give the desired compound (2) (2.75 g, 67%). ¹H-NMR (CD₃OD) δ: 1.64 (m, 1H), 1.82 (m, 1H), 2.01 (dd, J=18.0, 4.6 Hz, 1H), 2.24 (dd, J=18.0, 4.6 Hz, 1H), 2.82 (s, 1H), 2.92 (m, 1H), 2.96-3.01 (m, 2H).

### Reference Example 2

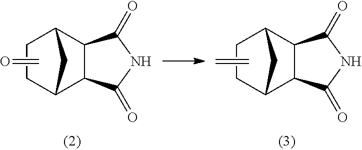

To a solution of methyl triphenylphosphonium bromide (11.43 g, 32 mmol) in tetrahydrofuran (100 ml) was added 1.5 mol/l n-butyllithium/hexane (23 ml, 32 mmol) at room temperature under nitrogen atmosphere. The mixture was stirred at 50° C. for 2 hours, and then Compound (2) (1.79 g, 10 mmol) was added thereto at 50° C. Subsequently, the mixture was refluxed for 16 hours. The reaction mixture was cooled to room temperature, and then water was added thereto. The mixture was extracted with ethyl acetate, and the organic layer was washed with water, dried over sodium sulfate. And the solvent was removed in vacuo. The residue was purified by a silica gel chromatography (hexane:ethyl acetate=100:0-70:30) to give the desired Compound (3) (0.86 g, 49%).

¹H-NMR (CDCl₃) δ: 1.39-1.50 (m, 2H), 2.04 (m, 1H), 2.33 (m, 1H), 2.76 (d, J=7.1 Hz, 1H), 2.82 (d, J=7.1 Hz, 1H), 2.84 (d, J=3.9 Hz, 1H), 3.15 (s, 1H), 4.80 (s, 1H), 5.08 (s, 1H), 8.32 (brs, 1H).

### Reference Example 3

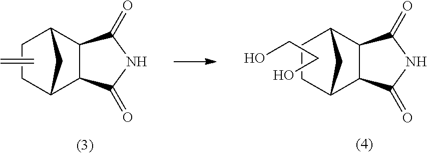

To a solution of Compound (3) (177 mg, 1 mmol) in a mixture of acetone (6 ml)/water (0.6 ml) was added 4-methylmorpholine oxide (176 mg, 1.5 mmol) and microencapsulated osmium (VIII) tetraoxide (120 mg) under nitrogen atmosphere. The mixture was stirred at room temperature for 64 hours. The reaction mixture was filtrated through Celite and washed out with methanol. The filtrate was concentrated in vacuo. The residue was purified by a silica gel chromatography (chloroform:methanol=100:0-80:20) to give the desired Compound (4) (190 mg, 90%).

¹H-NMR (CD₃OD) δ: 1.23 (m, 1H), 1.34 (dd, J=13.4, 2.3 Hz, 1H), 1.57 (dd, J=13.4, 4.9 Hz, 1H), 1.90 (m, 1H), 2.54-2.60 (m, 3H), 2.93 (d, J=6.7 Hz, 1H), 3.55 (d, J=2.3 Hz, 2H).

### Reference Example 4

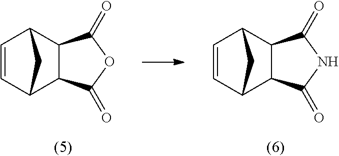

30% Aqueous ammonia was added to Compound (5), and the mixture was refluxed at 80° C. for 117 hours. The reaction mixture was concentrated in vacuo. The residue was crystallized from ethanol, and the crystal was removed out through a filter. The filtrate was concentrated in vacuo. The residue was washed with chloroform, and concentrated in vacuo. The resulting compound (6) was used in the next reaction without further purification (1.16 g, 71%).

¹H-NMR (CD₃OD) δ: 1.40 (m, 1H), 1.52 (m, 1H), 2.70 (d, J=1.6 Hz, 2H), 3.16 (m, 2H), 6.30 (m, 2H).

### Reference Example 5

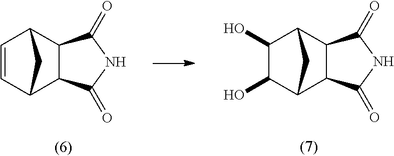

To a solution of Compound (6) (163 mg, 1 mmol) in a mixture of acetone (6 ml)/water (0.6 ml) was added 4-methylmorpholine oxide (176 mg, 1.5 mmol) and osmium (VIII) tetraoxide (5 mg, 0.012 mmol) under nitrogen atmosphere. The mixture was stirred at room temperature for 4 hours. The reaction mixture was concentrated in vacuo, and the residue was extracted with a mixture of ethyl acetate/tetrahydrofuran (1:1). The organic layer was washed with 20% aqueous sodium hydrosulfite, dried over sodium sulfate. And the solvent was removed in vacuo. The residue was washed with chloroform, and the resulting crystal was filtered to give the desired Compound (7) (96 mg, 49%).

¹H-NMR (DMSO-d₆) δ: 0.91 (d, J=11.3 Hz, 1H), 1.67 (d, J=11.3 Hz, 1H), 2.19 (s, 2H), 2.52 (s, 2H), 3.61 (d, J=1.3 Hz, 2H), 4.85 (brs, 2H), 11.30 (brs, 1H).

### Reference Example 6

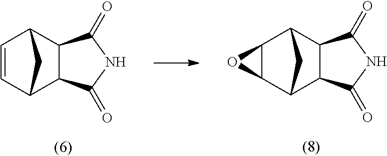

To a solution of Compound (6) (1.15 g, 7 mmol) in dichloromethane (30 ml) was added meta-chloroperbenzoic acid (1.91 g, 11 mmol) at ice temperature under nitrogen atmosphere. The mixture was stirred at room temperature for 16 hours. The resulting crystal was filtered and washed with water and ether to give the desired Compound (8) (0.81 g, 65%).

¹H-NMR (DMSO-d₆) δ: 0.63 (d, J=11.1 Hz, 1H), 1.17 (d, J=11.1 Hz, 1H), 2.70 (s, 2H), 2.77 (s, 2H), 3.37 (s, 2H), 11.30 (brs, 1H).

### Reference Example 7

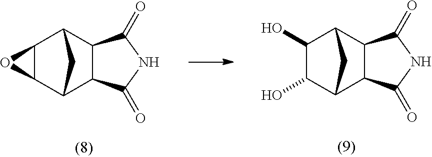

5% Aqueous sulfuric acid was added to Compound (8) (179 mg, 1 mmol), and the mixture was stirred at 60° C. for 4 hours. The reaction mixture was concentrated in vacuo, and the residue was purified by a silica gel chromatography (chloroform:methanol=100:0-80:20) to give the desired Compound (9).

¹H-NMR (CD₃OD) δ: 1.88-1.94 (m, 2H), 2.50 (m, 1H), 2.59 (m, 1H), 3.00 (m, 1H), 3.10 (dd, J=9.4, 5.9 Hz, 1H), 3.83 (m, 1H), 4.11 (m, 1H).

### Reference Example 8

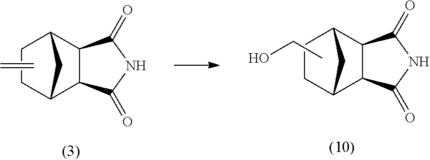

To a solution of Compound (3) (177 mg, 1 mmol) in tetrahydrofuran (10 ml) was added 1 mol/l borane/tetrahydrofuran (1 ml, 1 mmol) at room temperature under nitrogen atmosphere. The mixture was stirred for 2 hours, and then water was added thereto. 2 mol/l Aqueous sodium hydroxide and 30% aqueous hydrogen peroxide were added thereto, and the mixture was stirred at room temperature for 1 hour. Water was added to the reaction mixture. The mixture was extracted with a mixture of ethyl acetate/tetrahydrofuran (1:1). The organic layer was washed with 10% aqueous sodium hydrogen sulfite and brine, dried over sodium sulfate. And the solvent was removed in vacuo. The residue was purified by a silica gel chromatography (chloroform:methanol=100:0-80:20) to give the desired Compound (10) (20 mg, 10%).

### Example 1

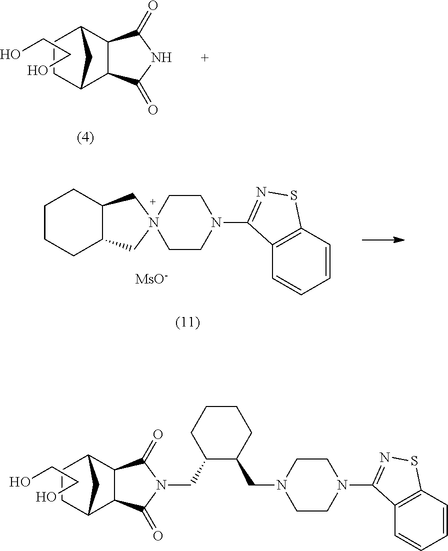

To a solution of Compound (4) (70 mg, 0.33 mmol) and Compound (11) (119 mg, 0.28 mmol, prepared by the method described in Patent Reference 2) in dimethylformamide (2.4 ml)/toluene (6 ml) was added potassium carbonate (77 mg, 0.56 mmol) under nitrogen atmosphere. The mixture was stirred at 100° C. for 17 hours, and then cooled to room temperature. The re-action mixture was poured into ice/water, extracted with chloroform and dried over sodium sulfate. And the solvent was removed in vacuo. The residue was purified by a silica gel chromatography (chloroform:methanol=100:0-90:10). The resulting compound was purified by a recycle HPLC (chloroform) to give the desired compound (100 mg, 66%).

¹H-NMR (CDCl₃) δ: 0.99-1.74 (m, 12H), 1.87 (d, J=11.5 Hz, 1H), 1.93 (d, J=11.5 Hz, 1H), 2.22 (dd, J=12.5, 6.2 Hz, 1H), 2.53 (d, J=7.0 Hz, 1H), 2.56-2.70 (m, 6H), 2.73 (d, J=4.2 Hz, 1H), 2.83 (d, J=7.0 Hz, 1H), 3.13-3.41 (m, 2H), 3.45-3.57 (m, 4H), 3.65 (s, 2H), 3.94 (d, J=11.5 Hz, 1H), 7.35 (t, J=8.0 Hz, 1H), 7.46 (t, J=8.0 Hz, 1H), 7.80 (d, J=8.0 Hz, 1H), 7.90 (d, J=8.0 Hz, 1H).

The following Examples 2-6 were prepared in a similar manner to Example 1.

### Example 2

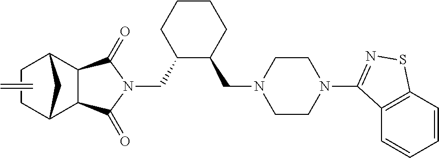

¹H-NMR (CDCl₃) δ: 0.94-1.30 (m, 5H), 1.33-1.44 (m, 2H), 1.45-1.62 (m, 2H), 1.62-1.72 (m, 2H), 1.88 (m, 1H), 2.05 (m, 1H), 2.23 (dd, J=12.3, 6.6 Hz, 1H), 2.32 (m, 1H), 2.58-2.78 (m, 7H), 2.83 (d, J=3.8 Hz, 1H), 3.14 (s, 1H), 3.33 (dd, J=12.3, 10.4 Hz, 1H), 3.46-3.59 (m, 4H), 3.95 (dd, J=13.0, 3.8 Hz, 1H), 4.79 (s, 1H), 5.08 (s, 1H), 7.35 (t, J=8.0 Hz, 1H), 7.46 (t, J=8.0 Hz, 1H), 7.80 (d, J=8.0 Hz, 1H), 7.91 (d, J=8.0 Hz, 1H).

### Example 3

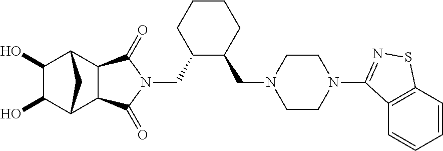

¹H-NMR (CDCl₃) δ: 0.93-1.76 (m, 12H), 1.86 (m, 1H), 2.23 (dd, J=12.3, 6.2 Hz, 1H), 2.53 (s, 2H), 2.57-2.70 (m, 7H), 2.94 (brs, 1H), 3.33 (dd, J=13.2, 10.4 Hz, 1H), 3.52 (brs, 4H), 3.87 (s, 2H), 3.98 (dd, J=13.2, 3.7 Hz, 1H), 7.36 (t, J=7.8 Hz, 1H), 7.46 (t, J=7.8 Hz, 1H), 7.81 (d, J=7.8 Hz, 1H), 7.91 (d, J=7.8 Hz, 1H).

### Example 4

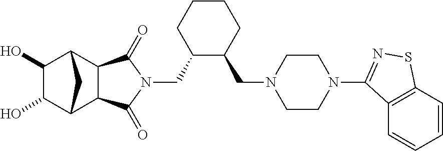

¹H-NMR (CDCl₃) δ: 0.93-1.64 (m, 9H), 1.74-1.81 (m, 3H), 2.24 (dd, J=12.7, 5.8 Hz, 1H), 2.53-2.73 (m, 6H), 2.79 (brt, J=4.5 Hz, 1H), 2.93 (m, 1H), 3.04 (m, 1H), 3.32 (dd, J=12.7, 10.5 Hz, 2H), 3.44-3.60 (m, 4H), 3.67-4.02 (m, 3H), 4.18 (s, 1H), 7.35 (t, J=7.8 Hz, 1H), 7.46 (t, J=7.8 Hz, 1H), 7.80 (d, J=7.8 Hz, 1H), 7.91 (d, J=7.8 Hz, 1H).

### Example 5

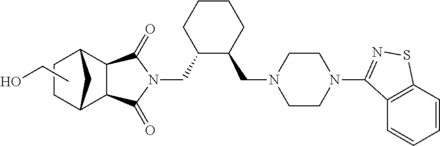

¹H-NMR (CDCl₃) δ: 0.85 (m, 1H), 0.93-1.44 (m, 7H), 1.44-1.60 (m, 2H), 1.66 (d, J=11.2 Hz, 2H), 1.77-2.30 (m, 5H), 2.52-2.73 (m, 7H), 2.78 (m, 1H), 2.93 (d, J=7.1 Hz, 1H), 3.33 (dd, J=13.1, 10.7 Hz, 1H), 3.41-3.61 (m, 5H), 3.70 (dd, J=10.7, 6.5 Hz, 1H), 3.94 (dd, J=13.1, 3.2 Hz, 1H), 7.35 (t, J=8.0 Hz, 1H), 7.46 (t, J=8.0 Hz, 1H), 7.80 (d, J=8.0 Hz, 1H), 7.91 (d, J=8.0 Hz, 1H).

### Example 6

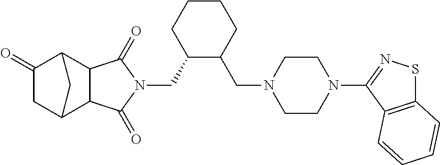

¹H-NMR (CDCl₃) δ: 0.98-1.26 (m, 5H), 1.39-1.88 (m, 6H), 2.03 (dd, J=4.4, 18.1 Hz, 1H), 2.23-2.29 (m, 2H), 2.64 (m, 5H), 2.91 (q, J=7.1 Hz, 2H), 3.05 (m, 1H), 3.14 (m, 1H), 3.46 (dd, J=10.4, 13.2 Hz, 1H), 3.52 (m, 4H), 4.06 (dd, J=4.4, 13.7 Hz, 1H), 7.35 (t, J=7.2 Hz, 1H), 7.46 (t, J=7.2 Hz), 7.80 (d, J=7.2 Hz, 1H), 7.90 (d, J=7.2 Hz, 1H).

### Test 1. Anti D2 Activity (Receptor Binding Test)

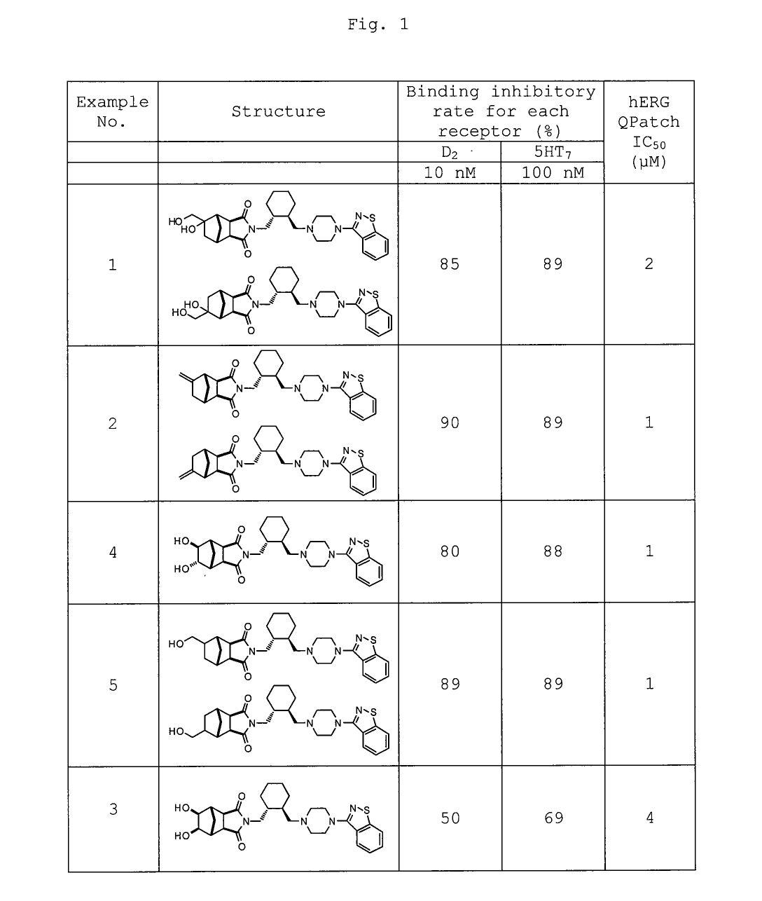

According to the known method (e.g. Japan. J. Pharmacol., 53, 321-329 (1990)), the above-captioned experiment was carried out using [3H] spiperone, i.e., the binding amount of [3H] spiperone to the preparation cell membrane expressing human D2 receptor was measured, and then the binding inhibitory rate by the test compound (10 nM) was measured/calculated. The results are shown in FIG. 1. Higher binding inhibitory rate means higher affinity of the test compound for human D2 receptor.

### Test 2. Anti 5HT₇ Activity (Receptor Binding Test)

To a buffer solution containing 50 mM Tris-HCl (pH=7.6), 4 mM CaCl₂ and 0.5 mM EDTA were added 50 μl of [³H]5-CT (final concentration: 0.5 nM), 1 μl of a solution of the test compound, and 150 μl of h5-HT₇/CHO membrane preparation (the total amount of the reaction solution: 201 μl). Using the reaction solution, the receptor binding activity of each test compound was measured. The reaction solution was incubated at room temperature for 40 minutes, and then the reaction solution was quickly filtrated under reduced pressure through a glass-fiber filter. The glass-fiber filter was washed twice with 200 μl of 50 mM Tris-HCl (pH=7.6), and then put into a counting vial containing 4 ml of ACS-II (Amersham), and the receptor binding radioactivity of the residual on the filter was measured with a liquid scintillation counter. And the radioactivity was also measured in the presence of 10 μM SB-269970 (5-HT₇ receptor ligand). Based on the above results, the binding inhibitory rate of each test compound (100 nM) was calculated. The results are shown in FIG. 1. Higher binding inhibitory rate means higher affinity of the test compound for human 5HT₇ receptor.

Test 3. Evaluation for hERG Potassium Channel (Side Effect)

The effect of each example compound for hERG (human ether-a-go-go-related gene) potassium channel current was evaluated through electrophysiological experiments using an automated patch-clamp system.

### Preparation of Test Solution

The test solution of each example compound was prepared at 20 mM using DMSO, and then frozen. The test solution was unfrozen just before use, and stepwise dilution of the test solution to an appropriate concentration was conducted with the extracellular solution.

### Cell Line

A CHO cell line stably expressing hERG potassium channel was supplied by Sophion Bioscience A/S and used in the following experiments.

### (Solutions)

Extracellular solution (mM): 2 CaCl₂, 1 MgCl₂, 10 HEPES, 4 KCl, 145 NaCl, 10 glucose.
Intracellular solution (mM): 5.4 CaCl₂, 1.8 MgCl₂, 10 HEPES, 31 KOH, 10 EGTA, 120 KCl, 4 ATP.

### Equipment and Electrophysiological Recording

The hERG potassium channel current was recorded by the whole-cell patch clamp method using an automated planar patch-clamp system QPatch HT (Sophion Bioscience A/S). Patch-clamp experiments were performed in voltage-clamp mode and the following stimulation protocol was applied to the experiments. The membrane potential was held at −80 mV, and then depolarized to +20 mV for 5 seconds after adjusting the pulse to −50 mV for 20 milliseconds to define the baseline, followed by repolarization step to −50 mV for seconds to evaluate the tail current amplitude. The stimulation was repetitively given every 15 seconds. Experiments were conducted at room temperature (22±2° C.).

### Data Analysis

To evaluate the effect of each example compound, the fraction of the tail current amplitude was calculated by averaging 3 data points of initial control current and 3 data points of remaining current 5 minutes after each application of the examples. The effects of each example were determined from cumulative applications of the increasing 4 concentrations and calculated as a percent of the blocked current (inhibition [%]). The data points were fitted with Hill equation to calculate 50% inhibition concentrations (IC₅₀ [μM]). The results are shown in FIG. 1.

1. A cycloalkane derivative of formula [1]: 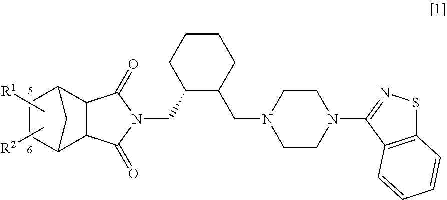
wherein
R¹ and R² are attached at C5 or C6 of the bicycloheptane dicarboximide ring (at the same carbon atom or different carbon atoms), and are independently hydrogen, hydroxyl, hydroxymethyl or —CH(C₁₋₃ alkyl)OH,
provided that when one of R¹ and R² is hydrogen, the other is neither hydrogen nor hydroxyl, or
when R¹ and R² are attached at the same carbon atom, they may be combined to form ═O, ═CH₂ or ═CH—C₁₋₃ alkyl,
or a stereoisomer or an acid addition salt thereof.

2. The cycloalkane derivative of claim 1 wherein the compound of formula [1] is the following stereo structure: 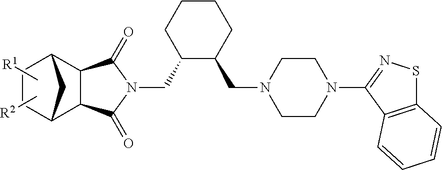
or a stereoisomer or an acid addition salt thereof.

3. The cycloalkane derivative of claim 1 or 2 wherein R¹ and R² is independently hydroxyl, hydroxymethyl or —CH(C₁₋₃ alkyl)OH, or a stereoisomer or an acid addition salt thereof.

4. The cycloalkane derivative of claim 1 or 2 wherein one of R¹ and R² is hydrogen, and the other is hydroxymethyl or —CH(C₁₋₃ alkyl)OH, or a stereoisomer or an acid addition salt thereof.

5. The cycloalkane derivative of claim 1 or 2 wherein R¹ and R² are attached at the same carbon atom and combined to form ═O, ═CH₂ or ═CH—C₁₋₃ alkyl, or a stereoisomer or an acid addition salt thereof.

6. The cycloalkane derivative of claim 1 wherein the compound of formula [1] is represented by the following structure: 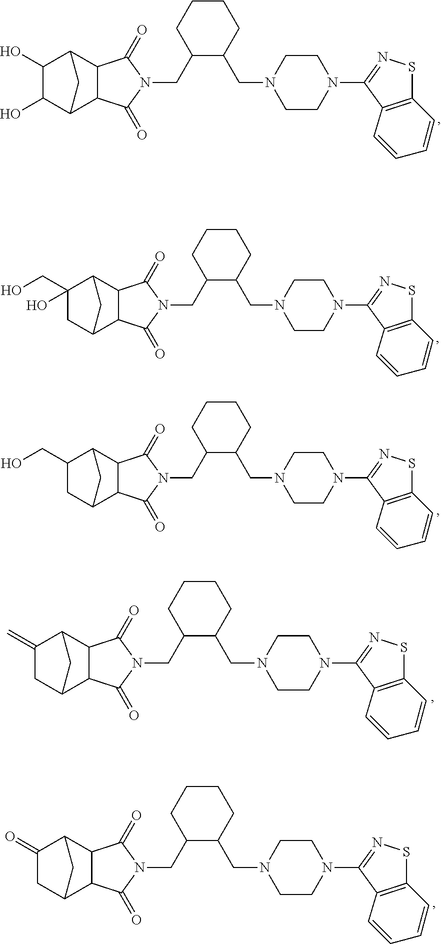
or a stereoisomer or an acid addition salt thereof.

7. The cycloalkane derivative of claim 1 wherein the compound of formula [1] is represented by the following structure: 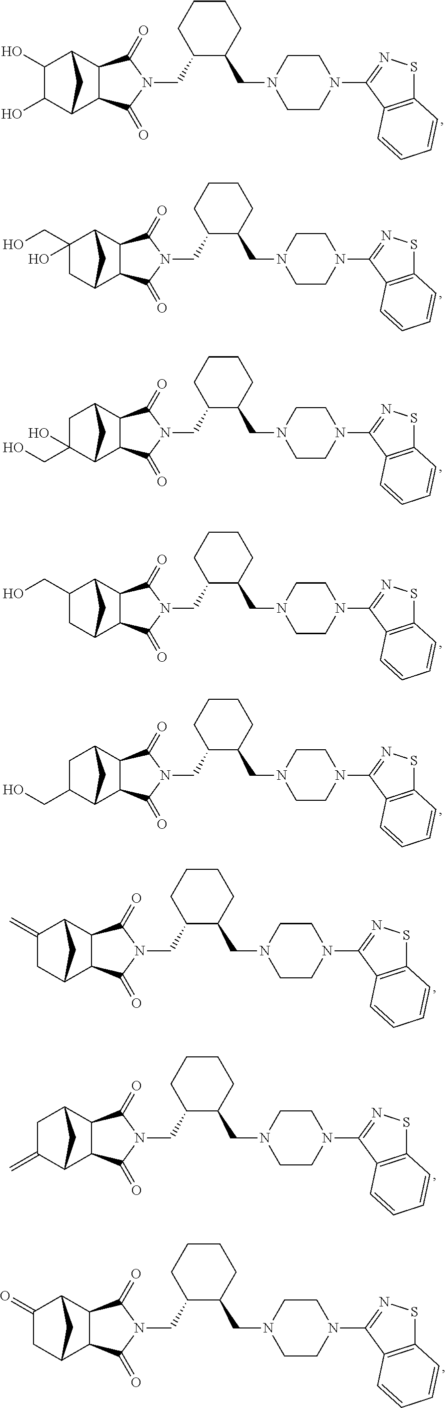 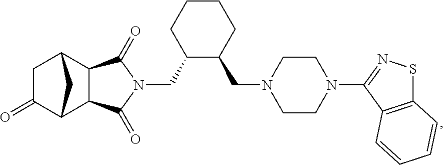
or a stereoisomer or an acid addition salt thereof.

8. An antipsychotic agent comprising the cycloalkane derivative of claim 1 or a stereoisomer or an acid addition salt thereof.

9. A method for treating psychosis comprising administering an effective amount of the cycloalkane derivative of claim 1 or a stereoisomer or an acid addition salt thereof to a mammal in need thereof.

10. Use of the cycloalkane derivative of claim 1 or a stereoisomer or an acid addition salt thereof in preparation of an antipsychotic agent.
